(19) United States
(12) Patent Application Publication
(10) Pub. No.: US 2017/0074788 A1
(43) Pub. Date: Mar. 16, 2017

(54) SPECTROSCOPY METHOD AND SYSTEM

(71) Applicants: NIKOLAUS WELLNER, NORWICH NORFOLK (GB); KATE KEMSLEY, NORWICH NORFOLK (GB)

(72) Inventors: NIKOLAUS WELLNER, NORWICH NORFOLK (GB); KATE KEMSLEY, NORWICH NORFOLK (GB)

(73) Assignee: INSTITUTE OF FOOD RESEARCH, NORWICH NORFOLK (GB)

(21) Appl. No.: 15/270,643

(22) Filed: Sep. 20, 2016

Related U.S. Application Data
(63) Continuation-in-part of application No. PCT/GB2015/050854, filed on Mar. 23, 2015.

(30) Foreign Application Priority Data
Mar. 24, 2014 (GB) 1405223.7

(51) Int. Cl. G01N 21/359; A01G 1/00; A01G 7/04; G01N 33/00

(52) U.S. Cl. CPC G01N 21/359; G01N 33/0098; A01G 1/001; A01G 7/045

(57) ABSTRACT

We describe a method of making a prediction of when a tuber will sprout. In some embodiments the method comprises making a measurement of an optical reflectance of the tuber in an eye region of the tuber; making a reference measurement of optical reflectance of the or another tuber, processing the tuber eye measurement in combination with the reference measurement; and making a prediction of when the tuber will sprout in response.

The present invention is a Continuation-in-Part application of Serial No. PCT/GB2015/050854, filed on 23 Mar. 2015; which claims priority from GB 1405223.7, filed 24 Mar. 2014, the entirety of both of which are incorporated herein by reference.

### FIELD OF THE INVENTION

The invention generally relates to methods and systems for predicting sprouting in root vegetables, in particular potato tubers, using Visible/Near-infrared spectroscopy.

### BACKGROUND TO THE INVENTION

The quality and composition of potatoes and other vegetables has previously been evaluated using a wide range of techniques, including spectroscopic techniques:

M. W. Young et al. estimated the nitrogen concentration in potato tissues using near-infrared reflectance spectroscopy (M. W. Young et al., Potato Research 40 (1997), p. 215-220). Furthermore, as outlined in N. U. Haase, Starch 58 (2006), p. 268-273, near-infrared spectroscopy allowed to determine the phosphorous content of starch and protein content of potatoes. B. Singh used near-infrared spectroscopy to determine the water content in potato tubers (“Visible and Near-Infrared Spectroscopic Analysis of Potatoes”, Master thesis, McGill University, October 2005). General background material relating to potato tubers can be found in: Coleman, W. K., Physiological ageing of potato tubers: A review, Annals of Applied Biology 137(2) (2000), p. 189-199; Hartmann, A., et al., Reactivation of Meristem Activity and Sprout Growth in Potato Tubers Require Both Cytokinin and Gibberellin, Plant Physiology 155(2) (2011), p. 776-796; Sergeeva, L. I., et al., Starch-related enzymes during potato tuber dormancy and sprouting, Russian Journal of Plant Physiology 59(4) (2012), p. 556-564; and Suttle, J. C., Physiological regulation of potato tuber dormancy, American Journal of Potato Research 81(4) (2004), p. 253-262.

There is a general need for further improvements in the evaluation of root vegetable quality, in particular in the evaluation of tubers such as potato tubers.

### SUMMARY OF THE INVENTION

According to a first aspect of the present invention there is therefore provided a method of making a prediction of when a tuber will sprout, the method comprising: making a tuber eye measurement of an optical reflectance of said tuber in an eye region of the tuber; making a reference measurement of optical reflectance of the said or another tuber; processing said tuber eye measurement in combination with said reference measurement; and making a prediction of when said tuber will sprout responsive to a result of said processing.

Broadly speaking the inventors have determined by extensive experimentation that reliable predictions of tuber sprouting can be made based upon an optical reflectance spectrum of a bud, more particularly an apical bud of the tuber. Analysis of the spectral changes demonstrates that there are various wavelengths which can reliably be employed to predict when a tuber will sprout. This prediction may comprise, for example, a prediction of a number of days until sprouting or a prediction that sprouting will occur (or will not occur) after (or not before) a defined time interval, or within a defined time window.

We note that references to making a prediction of “when the tuber will sprout” are to be interpreted as including “when the tuber is about to sprout”. Furthermore, in embodiments predicting when a tuber will sprout may comprise, for example identifying when a gradient of the reflection intensity changes and/or determining when the reflection intensity (or integrated intensity) falls to or by a threshold value. Thus the point at which the tuber will sprout may be the point of intervention described further below.

Regression analysis has shown that there are various wavelengths which may be used to predict sprouting—for example in one cultivar a wavelength of about 690 nm provides information useful for predicting sprouting. In principle, a measurement at a single wavelength or range of wavelengths may be employed to predict sprouting or measurements at or over a plurality of wavelengths may be employed.

Thus in embodiments a wavelength-sensitive optical sensing system may be employed to measure features of an optical reflectance spectrum of the eye region of the tuber to obtain the tuber eye and reference measurements. This may be an optical reflectance spectrometer/probe, gathering reflectance data over a range of wavelengths, or it may be a simpler system, merely interrogating two, three or a few wavelengths or wavelength ranges, for example using one or more filters on the optical source/detector. In principle (and this is contemplated by aspects of the invention) the optical reflectance measurement may be supplemented or replaced by an optical absorbance measurement, although this is generally less practical. Nonetheless aspects and embodiments of the invention contemplate a method/system as described herein in which the optical reflectance measurements are supplemented or substituted by optical absorbance measurements.

The skilled person will appreciate that “optical” is not limiting with respect to the wavelength(s) used. In particular, “optical” may refer to one or more of the UV, visible, infrared/near-infrared, microwave, or other parts of the spectrum. Hence, “optical” is not to be understood as limiting with respect to the wavelength(s) wherever used in this description.

The reference measurement may in some embodiments comprise a measurement at an eye-region of the tuber. This may be the same eye at which the above-specified “tuber eye measurement” is performed, or it may be a different eye on the same tuber, or it may be an eye on a different tuber.

In preferred embodiments the reference measurement is a measurement at another location, in particular a non-eye region, of the or another tuber. This may be a measurement at one or more wavelengths/wavelength ranges; for example in some preferred embodiments a ratio of spectra (for the eye-region measurement and reference measurement) is determined. (We describe later some advantages of measuring at multiple different wavelengths in multiple different locations on one or more tubers). In other approaches the reference measurement may additionally or alternatively be an earlier measurement made on the or another tuber, in effect providing a base line from which a change can be judged. In still other approaches, in principle the reference measurement may be a measurement at a different wavelength to the tuber eye measurement, but potentially made in the same place (the tuber eye) although this is more prone to measurement noise. Again, in principle, a range of different approaches may be employed together—for example using data at multiple wavelengths (a spectrum), from an eye region and a non-eye region, optionally repeating measurements on the same or different tubers over time.

In preferred embodiments a ratio of eye-region and reference measurements, at a single wavelength or over a plurality of wavelengths, is used in a regression model, in particular a continuum regression model, to provide a prediction in the form y=Kx where y is a prediction of time to sprouting, K is a scalar or vector constant, and x is a scalar or vector wavelength ratio or spectrum ratio.

As previously alluded to, multivariate analysis, in particular partial least squares (PLS) analysis has identified a number of different wavelengths useful for detecting the onset of sprouting in tubers. In the PLS analysis wavelengths with particularly large (either positive or negative) regression coefficients are wavelengths which are particularly useful for predicting sprouting. For example, depending upon the cultivar the most salient feature appears to be at a wavelength in the range 600 nm to 750 nm, for example at 640 nm+/−50 nm, 675 nm+/−50 nm or 690 nm+/−50 nm. However these are not the only wavelengths which may be employed as can be seen, for example, from the graphs of regression coefficients presented later. Thus, for example, the range 550 nm-650 nm appears potentially useful, and/or the range 675 nm-750 nm, and/or the range 650 nm-700 nm. In embodiments the optical measurement is made at one or more wavelengths in the range 300 nm-2000 nm, more particularly 500 nm-1500 nm.

Some preferred embodiments of the method include making a further optical reflectance measurement, preferably but not essentially at one or more of the same wavelengths as the previously described tuber eye measurement and reference measurement, making this measurement on a ‘background’ that is non-eye region of the or another tuber. This is useful for acquiring reference data for the tuber(s) being measured and also for compensating for any potential effects arising from interference from background ambient lighting. (Such an approach is particularly helpful in a multivariate analysis method, for example partial least squares, where an interfering component with a large variance can result in a prediction error.

In some embodiments measurements at multiple positions on a tuber at multiple wavelengths may be made by a colour or hyperspectral camera, and the necessary data extracted from the captured image(s). The skilled person will understand that the camera may be sensitive to one or more wavelengths or ranges of wavelengths, such as, but not limited to wavelengths in the UV, visible, infrared/near-infrared, microwave, or other parts of the spectrum.

The skilled person will appreciate that there are many ways in which the tuber eye measurement may be processed in combination with the reference measurement. For example a comparison may be made between these two measurements, by determining a difference between the measurements and/or a ratio of the measurements. However in some preferred approaches the processing of the tuber eye reference measurements comprises providing these measurements to a mathematical model which has been trained using a training data set defining actual measurement time to sprouting or a set of corresponding measurements. Again the skilled person will appreciate that there are many such mathematical models which may be employed including, but not limited to: multiple linear regression, principle component analysis/regression, partial least squares analysis/regression, various types of artificial neural network, and many related (supervised) machine learning techniques including, but not limited to, Bayesian techniques including Bayesian networks, clustering techniques, support vector machines, and the like. Optionally, but not necessarily, a set of mathematical models may be trained one for each of a set of cultivars, so that the appropriate model for a particular cultivar may be selected, for increased accuracy. In one preferred approach the model comprises a partial least squares model. As the skilled person will be aware, in broad terms in PLS a training set is provided, for example of spectral data (high-dimensional data) and one or more variables, such as time to sprouting and the PLS system builds a linear relationship between the spectra and time for sprouting that is then used for prediction of time to sprouting with new spectral data. This approach is well known as a technique for analysing spectral data and, as the skilled person will know, there are many software packages and libraries available to implement such a technique. Although, typically, such a technique employs high-dimensional data, that is a spectrum measured at many different wavelength points, it is not restricted to this and may be used with lower dimensional data.

In embodiments the optical reflectance measurement data may be pre-processed, for example compensating for a reference background signal level as previously described and/or processing the data so that it is mean-centred (average reflectance of zero with peaks above and below this level). The latter is particularly helpful for PLS. The skilled person will be aware of many potential pre-processing techniques which may be employed.

In a related aspect the invention also provides a method of identifying a potato or batch of potatoes for sale or use employing the techniques described above to make a prediction of when the potato or one or more potatoes of the batch will sprout, then making a decision on sale or use of the batch in response to the prediction.

In a further related aspect the invention provides a method of processing potatoes, the method comprising: making a tuber eye measurement of an optical reflectance of a potato tuber in an eye region of the tuber; making a reference measurement of optical reflectance of the said or another tuber; making a prediction of when said tuber will sprout responsive to said tuber eye measurement in combination with said reference measurement; and processing said potatoes responsive to said prediction, wherein said processing includes at least separating potatoes predicted to sprout sooner from potatoes predicted to sprout relatively later.

Embodiments of this technique may thus be employed to distinguish between potatoes predicted to sprout sooner than others, for example for sorting potatoes or distinguishing between batches of potatoes. For example potatoes predicted to sprout sooner than a particular deadline, say a number of days hence, may be identified for more rapid sale or use than others. Potatoes selected by the processing may be, for example, stored in a selected storage shed and/or dispatched for use.

In a further related aspect the invention provides an apparatus for making a prediction of when a tuber will sprout, the apparatus comprising: an optical reflectance measurement system for making a tuber eye measurement of an optical reflectance of an eye region of the tuber, and for making a reference measurement of optical reflectance of the said or another tuber; and a data processor, coupled to said optical reflectance measurement system, to process said tuber eye measurement in combination with said reference measurement and to make a prediction of when said tuber will sprout responsive to a result of said processing.

In some embodiments, a controlled illumination box may be exploited to perform optical reflectance measurements at an eye region of a tuber. As this allows for a controlled illumination of the tuber, a reference measurement may be omitted because the background illumination is known and/or may be controlled using the illumination box. Sprouting may be predicted using the controlled illumination box by processing one or more reflectance measurements conducted at an eye region of a tuber.

In a further aspect of the invention there is therefore provided a method of making a prediction of when a tuber will sprout, the method comprising: making a tuber eye measurement of an optical reflectance of said tuber in an eye region of the tuber; processing said tuber eye measurement; and making a prediction of when said tuber will sprout responsive to a result of said processing. The tuber eye measurement may be a single tuber eye measurement at one or more wavelengths or a wavelength range, or it may be a plurality of tuber eye measurements each at one or more wavelengths or a wavelength range. Preferably, when a plurality of tuber eye measurements is performed, the measurements may be averaged in order to make a prediction of when the tuber will sprout.

In a preferred embodiment of the method and apparatus, making said prediction of when said tuber will sprout comprises identifying a change in gradient of said optical reflectance over time, and/or identifying a reduction in a value of said optical reflectance to below a threshold level or by a threshold amount, and making a prediction of a point of intervention to inhibit said sprouting.

According to another aspect of the present invention there is provided a method for predicting sprouting of one or more root vegetables having at least one bud, the method comprising: measuring a reflectance of at least one of said one or more root vegetables to obtain a reflectance spectrum; and applying a multivariate model to said reflectance spectrum to predict said sprouting of said one or more root vegetables.

Embodiments of this method facilitate predicting the onset of sprouting in potato tubers with a precision of +/−7 days with a high confidence, more particularly with a precision of +/−4 days with a high confidence.

In a preferred embodiment of the method, a plurality of reflectance measurements is performed at two or more locations on each of the investigated root vegetables. In particular, it is preferred to take measurements at at least one apical bud and at least one location which does not comprise an apical bud. Measurements taken on the smooth skin located away from the apical buds may then be used as a background reference signal with regard to measurements taken on one or more apical buds.

In a further preferred embodiment of the method, the multivariate model is a linear mathematical model, in particular a Partial Least Squares model.

The Partial Least Squares model may then be applied to predict the sprouting using a calculated regression coefficient obtained from a previous reflectance measurement.

It will be appreciated that it is preferred to perform the reflectance measurements in a non-contact, non-destructive way. Furthermore, the skilled person will understand that the reflectance spectra may be obtained using light with a range of optical wavelengths, for example between 500 nm and 1200 nm, and may preferably be varied depending on the type and properties of the vegetable to be investigated.

The invention further provides a system for predicting sprouting of one or more root vegetables having at least one bud, the system comprising: a spectrometer to measure a reflectance of at least one of said one or more root vegetables to obtain a reflectance spectrum; and a processor configured to apply a multivariate model to said reflectance spectrum to predict said sprouting of said one or more root vegetables.

In a related aspect of the invention, there is provided a method for predicting sprouting of one or more root vegetables, in particular embodiments of the methods described above, the method comprising: measuring a reflectance of at least one of said one or more root vegetables at a plurality of points in time to obtain a plurality of reflectance spectra; fitting a curve to each of said reflectance spectra; and comparing said fitted curves to predict said sprouting of said one or more root vegetables.

A change or occurrence of one or more features at a certain wavelength or wavelength range of the curves may appear at a point in time at which a root vegetable starts sprouting. This may be used to predict sprouting of the one or more root vegetables.

In a preferred embodiment, the fitted curves are polynomial curves.

In a further preferred embodiment, the method further comprises subtracting each of the curves from an initial reflectance spectrum of the plurality of reflectance spectra prior to said comparison. In another preferred embodiment, the method further comprises subtracting each of the measured reflectance spectra from an initial reflectance spectrum of the plurality of reflectance spectra to obtain a plurality of normalised reflectance spectra, and wherein the fitting comprises fitting a curve to each of the normalised reflectance spectra.

The polynomial fitting may be applied to the apical bud:background ratio spectra.

Preferably, measuring a reflectance comprises measuring a reflectance at an apical bud of a said root vegetable.

As outlined above, the measurements may be performed in a non-contact, non-destructive mode. The measurements may cover a wavelength range between 500 nm and 2300 nm.

In a further aspect the invention provides a method of monitoring tuber sprouting to predict a point of intervention to inhibit the sprouting, the method comprising: making a time series of optical measurements on said tuber at at least one wavelength to provide time series spatial data; processing said time series optical data to monitor the evolution over time of a spectral feature in said optical measurements; and predicting a point of intervention to inhibit the sprouting, from said evolution over time.

In preferred embodiments the optical measurements are made at a plurality of wavelengths (or over a range of wavelengths) encompassing one or more spectral features of the tuber, wherein the (one or more) spectral feature is an integrated spectral feature comprising a sum or integration over the plurality of wavelengths or wavelength range, and wherein the processing comprises monitoring the evolution of the integrated spectral feature over time. Making the optical measurements at a plurality of wavelengths or over a range of wavelengths helps to improve reliability of the measurements because the peak wavelength of an optical feature in the spectrum may change slightly over time.

Preferably the measurements comprise optical reflectance measurements for practicality. The measurements at multiple wavelengths/over a range of wavelengths are then preferably integrated to provide an integrated measure of the monitored spectral feature. This can help to improve reliability of the measurements.

In some preferred embodiments predicting the point of intervention to inhibit the sprouting comprises identifying a change in gradient of the evolution over time of the monitored spectral feature (or integrated data where integration is employed).

In preferred embodiments the identifying of the change in gradient comprises identifying a reduction in the gradient. Thus in embodiments the prediction comprises identifying when

$$\frac{d I}{d t} > C ,$$

where C is a threshold value less than 0, l represents a value of the spectral feature (an integrated value where integration is employed), and t represents time.

In a further preferred embodiment of the method, predicting the point of intervention comprises identifying a reduction in a value of the monitored spectral feature (or integrated data where integration is employed) to below a threshold level or by a threshold amount.

In some preferred embodiments of the method the tuber is a potato and optical (reflectance) measurements are made of one or more eyes of the potato, preferably at one or more wavelengths in the range 600 nm to 750 nm, for example at approximately 690 nm or a wavelength up to 50 nm to either side of 690 nm.

In some preferred embodiments the method includes inhibiting the sprouting of the tuber by applying a sprouting suppressant to the tuber in response to the point of intervention prediction. Sprouting suppressants include, but are not limited to, chlorpropham (CIPC), for example applied as a hot-fog, spearmint oil, 1,4-dimethylnapthalene (DMN), 3-decen-2-one, caraway oil, clove oil, and other suitable suppressants known to those skilled in the art. Additionally or alternatively, temperature control methods may be used to inhibit sprouting, and/or atmospheric control methods (using, e.g. ethylene), and/or in-field treatments (using, e.g. maleic hydrazide).

### Forced Response Based Techniques

Further experimental work by the inventors has resulted in a paradigm shift in the approach to predicting sprouting. The spectral measurements described above relate to the presence of chlorophyll in the tuber, more particularly the eye of the tuber, from where a green shoot will sprout. However after significant investigation the inventors have ascertained that the spectral changes seen were to a large degree driven by the measurement process. More particularly, when keeping a potato in the dark and taking it out at intervals to perform a spectral measurement, it appeared that substantially all the spectral change resulting from chlorophyll production was driven by the illumination during the measurement process. At first sight this appears to negate the validity of the whole procedure, but on closer investigation it was determined that the results nonetheless were apparently valid. In this context it should be noted that these are difficult experiments to perform, because the measurements need to take place over a number of months, spanning the natural sprouting process, and can only be performed once a year following the annual potato harvest.

From these investigations it appears that the propensity to sprouting can in fact be measured by driving the tuber to produce chlorophyll and/or sprout, and then measuring the response to this forced development. This represents a new paradigm in the measurement approach and, based on this new understanding, improved techniques have been devised.

According to another aspect of the invention there is therefore provided apparatus for determining the condition of a tuber, the system comprising: a light source to stimulate the tuber to promote chlorophyll production and/or sprouting, wherein said light source is configured to provide substantially continuous optical stimulation to the tuber; an optical instrument to measure an optical response of the tuber; a controller to control said optical instrument to make a time series of optical measurements on said tuber at intervals during said substantially continuous optical stimulation to determine a time evolution of an optical response of the tuber, wherein said time series evolution is determined over a period of less than twenty four, sixteen, twelve, eight, six or four, two or one hours; a data processor to analyse said time evolution of said optical response to determine a condition of the tuber.

In embodiments the optical instrument measures absorbance and/reflectance of the tuber, more particularly of an eye region of the tuber, at one or more wavelengths. Thus the optical instrument may comprise a spectrometer. In principle, however, other optical responses, such as fluorescence, may additionally or alternatively be measured.

In a preferred embodiment the optical response of the (eye region) of the tuber is measured at one or more wavelengths in the range 600 nm to 750 nm (the wavelength ranges may be as described previously). However in principle other wavelengths/wavelength bands may be employed—for example a blue chlorophyll wavelength/band in the range 400-500 nm may be employed. In embodiments the measured optical response may be within 50 nm of an absorption band of chlorophyll (as measured either in the tuber or in a solution, for example of ether). Preferably the time series evolution is determined over a period of less than eight hours.

The light source may be combined with the optical instrument or may be separate to the optical instrument. In the latter case there may be two light sources, one to measure, for example, a reflectance (spectrum) of the tuber, the other to stimulate the tuber.

The light source raises the illumination level of the tuber, more particularly the eye region of the tuber, above that of the ambient illumination level. Where the light source is incorporated into the optical instrument the light source may be arranged to provide substantially continuous optical stimulation whilst the controller controls the instrument to make measurements at intervals during this substantially continuous illumination. In embodiments the illumination level may be greater than 1,000 lux; preferably the illumination level is sufficiently bright to allow the evolution of the optical response of the tuber to be monitored for a period of less than 8, 6, 4, 2 or 1 hours.

Some preferred embodiments of the apparatus include a tuber holder to hold the sample tuber, in particular the eye region of the tuber, such that it is illuminated by the light source, and such that a sensor/sensor head/probe of the optical instrument can measure the optical response of the eye region. The holder may be configured to allow relative motion of one or more of the sample tuber, illumination source, and sensor. This facilitates obtaining a time series of measurements of the eye region of the tuber whilst illuminating the eye region.

In embodiments the data processor determines a sprouting propensity of the tuber, more particularly a prediction of when the tuber will sprout, although this may include (or be substituted by) a determination of a point of intervention to inhibit sprouting of the tuber. In broad terms the data processor determines a speed of response of the eye region of the tuber to the stimulation.

In embodiments the data processor analyses the optical response by determining one or more of: a time interval until a threshold change in the optical response, for example a threshold reduction in reflectance at one or more wavelengths; a rate of change of the optical response, for example a gradient of the reflectance over time; and a curve fit to the optical response. The skilled person would appreciate that when performing a curve fit one or more parameters are typically adapted to fit the curve; the sprouting propensity may then be determined from one or more of these parameters. The optical response may be a response at a particular single wavelength, or over a wavelength band, or may be, for example, an integrated area under part of the spectrum, or may be some other optical response.

In embodiments the data processor may comprise a suitably programmed general purpose computer system or a dedicated microcontroller or digital signal processor. Typically the data processor will include working memory, non-volatile program memory, and an interface to the optical system to capture optical measurements. The data processor may include an output device such as a display and/or network connection, to provide an indication of the sprouting propensity in any suitable format, such as a prediction of when the tuber will sprout or how long to predicted sprouting.

The program memory may store processor control code to control the data processor to capture optical measurements and to analyse the time evolution of the optical measurements to determine a condition of the tuber, as previously described. For example experiments have indicated that a curve of the general shape shown in FIG. 12b described later can be expected, that is comprising an initial substantially flat portion followed by a changing region, which may define an approximately linear change. The time lag to the changing portion may define the sprouting propensity (the greater the lag the lower the propensity). However there are other ways in which sprouting propensity may be determined, for example from the slope of the changing region or, more generally, from a curve fit as previously described. In some preferred embodiments bright illumination is employed to drive chlorophyll production over a relatively short period, for example of order an hour or less; measurements may be taken at intervals of less than every 5 minutes or less than every 1 minute, for example around every 30 seconds.

In embodiments the illumination may comprise broadband illumination, for example from an incandescent light. Optionally the light source may have a predominant blue output, for example with a maximum output in the wavelength range 400-500 nm. This may stimulate precursors to chlorophyll, to thereby promote chlorophyll production. As previously described the rate of production of chlorophyll is dependent upon the level (and character) of illumination. Furthermore the way in which a tuber, more particularly the eye region of a tuber, produces chlorophyll also appears to depend upon the propensity of the tuber to sprout, more particularly on the time interval to actual sprouting. The precise manner in which the time-evolution of stimulated chlorophyll production changes with sprouting propensity (number of days to sprouting) has yet to be precisely determined. However from initial observations the aforementioned time lag, gradient and overall curve shape appear all to be potentially usable for this prediction.

In a related aspect the invention provides a method of determining the sprouting propensity of a tuber, the method comprising: applying substantially continuous stimulation to an eye region of the tuber to drive said eye region of the tuber to develop at a faster than natural rate; making a time series of optical measurements on said eye region of said tuber at at least one wavelength to provide time series optical data to determine a time evolution of an optical response of said eye region of the tuber during said stimulation, wherein said time series evolution is determined over a period of less than twenty four, sixteen, twelve, eight, six or four, two or one hours; and determining a sprouting propensity of the tuber from said time evolution of said optical response.

Although preferred embodiments of the above described method and apparatus employ optical stimulation in principle chemical stimulation, such as a hormone or other chemical stimulant, may alternatively be employed.

The invention also provides a system comprising means for implementing the above described method.

### BRIEF DESCRIPTION OF THE DRAWINGS

These and other aspects of the invention will now be further described, by way of example only, with reference to the accompanying figures in which:

FIG. 1 shows an example experimental setup of an embodiment of the invention;

FIG. 2 shows a series of reflectance spectra of an apical bud;

FIG. 3 shows a set of visible/near-infrared data;

FIGS. 4a and 4b show cross-validation predictions and regression coefficients;

FIG. 5 shows further shows cross-validated predictions;

FIG. 6 shows regression coefficients for various types of potatoes;

FIG. 7 shows leave-tuber-out cross-validations;

FIG. 8 shows a boxplot summary of predictions;

FIG. 9 shows an example experimental setup of an embodiment of the invention;

FIGS. 10a to 10d show predicted sprouting age versus actual sprouting age for different types of tubers;

FIG. 11 shows regression coefficients related to PLS modelling for the Mozart tubers analysed as shown in FIG. 10a;

FIGS. 12a and 12b show visible IR spectra and time analysis of the spectra, respectively;

FIG. 13 shows calculated area versus sprouting age for Maris Piper tubers;

FIG. 14 shows calculated area versus sprouting age for different tubers;

FIGS. 15a and 15b show predicted sprouting age versus actual sprouting age for different Maris Piper tubers;

FIG. 16 shows Vis/NIR spectra of a Mozart tuber; and

FIGS. 17a and 17b show integrated feature intensity versus time for different tubers.

### DETAILED DESCRIPTION OF PREFERRED EMBODIMENTS

Sprouting is a major cause of losses, and therefore commercial losses, in stored potatoes. There is therefore a need for predicting the onset of sprouting in potato tubers.

In order to predict the onset of sprouting in potato tubers, visible/near-infrared spectra were collected using a portable, low-cost StellarNet spectrometer, equipped with a fibre-optic probe operating in the wavelength range of 500 nm to 1200 nm. The spectra were collected through a non-contact, non-destructive reflectance measurement, taken from the potato skin at two different locations: the apical buds (“eyes”) and a portion of the smooth skin located away from the eyes (“background”). In some experiments, pseudo-absorbance spectra were calculated for each eye, by ratioing to the previously collected background spectrum.

It was discovered that potatoes tend to age in a consistent manner which may be tracked by the visible/near-infrared spectra. A model based on a standard chemometric analysis method allowed predicting the length of time until sprouting of individual potatoes and potato batches.

Embodiments of the present invention were used to predict sprouting in various types of potatoes under different ageing conditions. In particular, studies related to monitoring of a Maris Piper potato forced into sprouting by exposure to light over a three-day period, longer-term monitoring of Maris Piper potatoes stored in the dark between analyses, and longer-term monitoring of Desiree and King Edward potatoes stored in the dark between analyses.

### Maris Piper Potato Exposed to Light Over a Three-Day Period

In a first attempt at detecting changes occurring before sprouting, an eye on a single Maris Piper tuber was monitored over a period of three days. Spectra were acquired hourly. This study took place at ambient laboratory temperature (21° C.) and the tuber was illuminated throughout, promoting quick sprouting. FIG. 1 shows an example experimental setup used in this investigation.

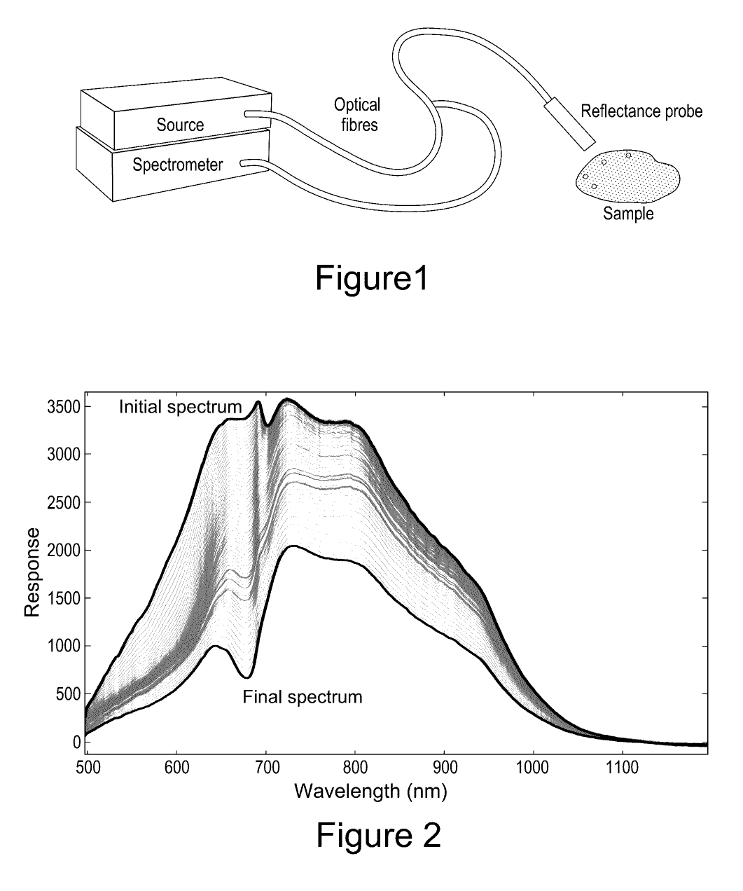

FIG. 2 shows a series of spectra obtained from an apical bud, collected over three days.

These data clearly show a profound difference between the initial (before sprouting) and final (after sprouting) spectra, and a gradual progression between the two states. This allowed predicting the onset of potato sprouting by pattern recognition methods applied to the “pre-sprouted” spectra.

### Longer-Term Monitoring of Maris Piper Potatoes Stored in the Dark Between Analyses

In a second series of experiments, 4 tubers were monitored every few days over a period of weeks. The tubers were from the 2012 harvest, and had been stored and treated with sprout suppressant as per standard commercial practice. Each tuber had a number of readily identifiable apical buds.

The tubers were stored at 4° C. in the dark between analyses. On each tuber, different sites were identified and labelled as follows: “site 0” corresponded to a background, and sites 1 to 3 were non-sprouted apical buds.

Replicate analyses (at least two-fold) were made of each site on each day of analysis, repositioning the fibre probe for each acquisition. This may be particularly useful as there may be a variance in the data arising from repositioning of the collection optics when using a handheld device.

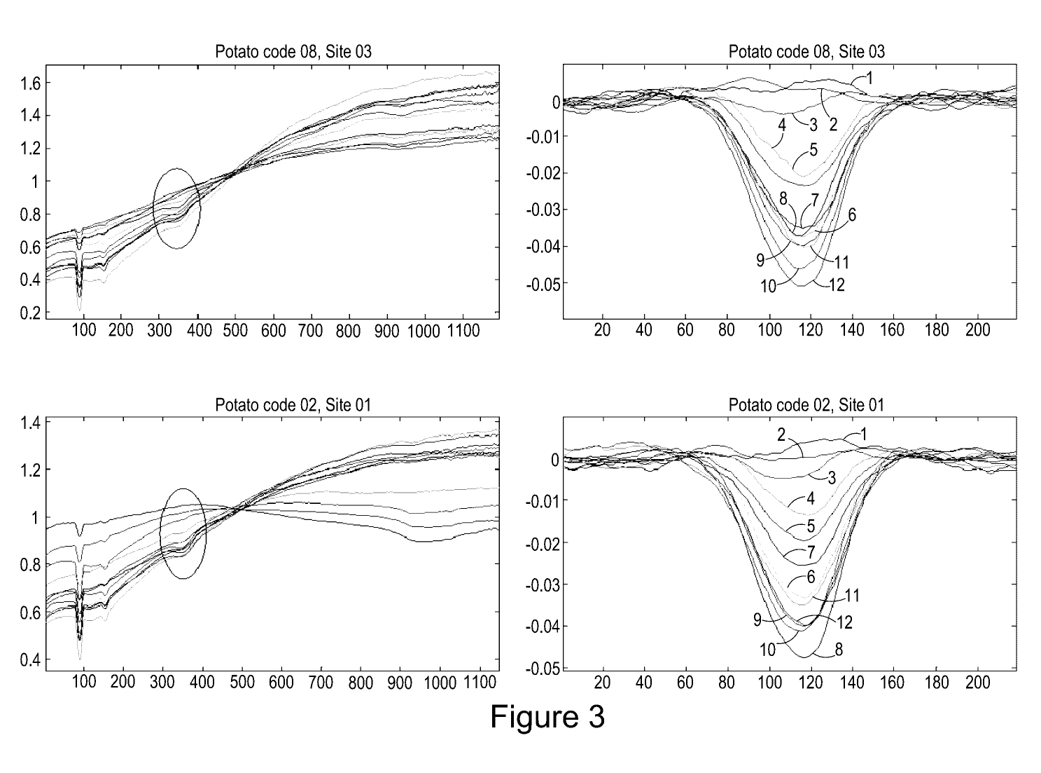

FIG. 3 shows a set of visible/near-infrared data from two eye sites on two different Maris Piper tubers which ultimately sprouted at around 4 weeks (here called analysis day 12). The data shown in these graphs relates to the ratio of tuber eye measurements and reference measurements taken on the respective tubers. In other words, background ambient light is taken into consideration. Note that in all cases the x-axes are in local data points, not wavelength units.

Left hand plots show the complete spectral range, ratioed to the site 0 backgrounds. Right hand plots show an expansion of the marked feature, which rapidly becomes prominent as the tubers age, in advance of the onset of sprouting.

For the majority of the sites monitored, a clear and systematic trend was seen in the spectra as the tubers aged, in advance of the visible signs of sprouting. As well as an overall spectral shape change, which represents an increasing difference in the visible/near-infrared reflectance of the eyes and the reference background skin, changes also occurred which led to the emergence of individual spectral bands in the spectra, specifically an increasingly negative feature at around 690 nm (data points 300-400 in the left hand figures). Background locations did not change quite so much, which is in agreement with literature studies which have shown that the dormancy-breaking chemical processes occur predominantly at the eyes.

The findings were consistent across different tubers, and persisted despite the large sampling variability that arose from the positioning of the fibre-optic probe relative to the tuber surface (replicate measurements were made to mitigate this source of variance, since a single measurement may be performed within a few seconds).

By using a “non-eye” location on the same tuber to acquire a reference background, other confounding factors such as ambient lighting may be accounted for.

In order to develope a predictive model for time until sprouting, a multivariate modelling may be applied to the visible/near-infrared spectra, in this example a Partial Least Squares modelling.

In this example, a continuum regression model is used to allow for making a prediction of when a tuber will sprout. The continuum regression model is of the form y=Kx, where y is a prediction of time until a tuber will sprout, K is a scalar or vector constant, and x represents a scalar or vector wavelength ratio or spectrum ratio.

The skilled person will appreciate that the continuum regression model used here is just one example of a model which may be exploited to make a prediction of when a tuber will sprout. The skilled person will immediately understand that any alternative model may be used to make this prediction.

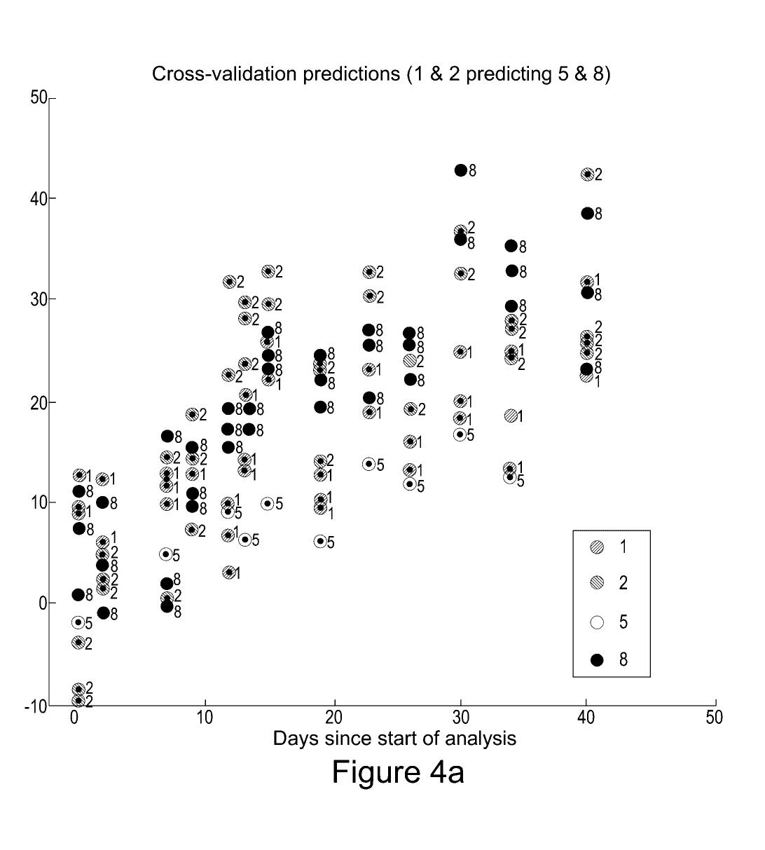

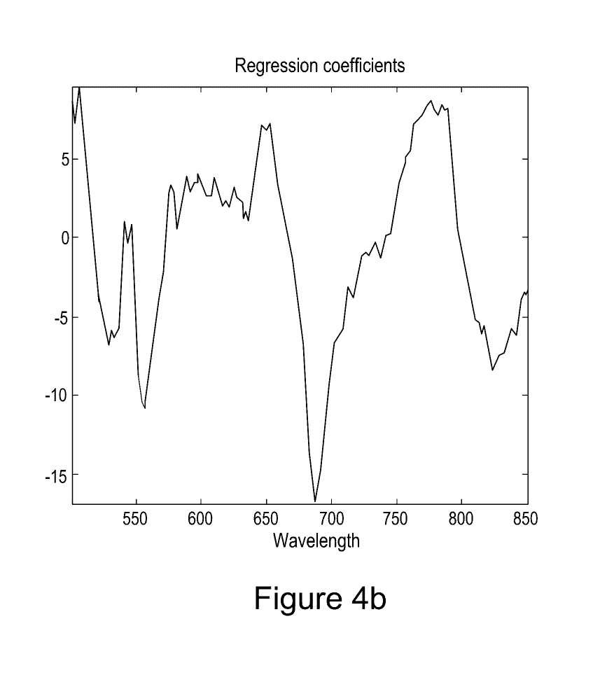

FIG. 4a shows cross-validated predictions of tuber age (data from tubers “1” and “2” predicting age of tubers “5” and “8”).

FIG. 4b shows regression coefficients as a function of wavelength for the data in FIG. 4a.

The modelling work showed that it was possible to apply Partial Least Squares modelling to the visible/near-infrared spectra from one collection of tubers, and use this model to predict the age of other tubers, as in this example Maris Piper potato tubers, from their spectra.

### Longer-Term Monitoring of Desiree and King Edward Potatoes Stored in the Dark Between Analyses

This study examined two further commercially important cultivars: King Edward and Desiree.

Three batches of potatoes were examined. The first batch was delivered direct from the commercial store soon after harvest. Six tubers were selected for study, three of each cultivar. The tubers were monitored using the same experimental protocol as described above in relation to the Maris Piper potatoes stored in the dark between analyses. In this instance, however, all potatoes were analysed until sprouting, and this was found to occur at somewhat different times for each tuber.

The visible/near-infrared spectra were analysed using Partial Least Squares regression, in this example using calculated “time until sprouting” as the dependent variate.

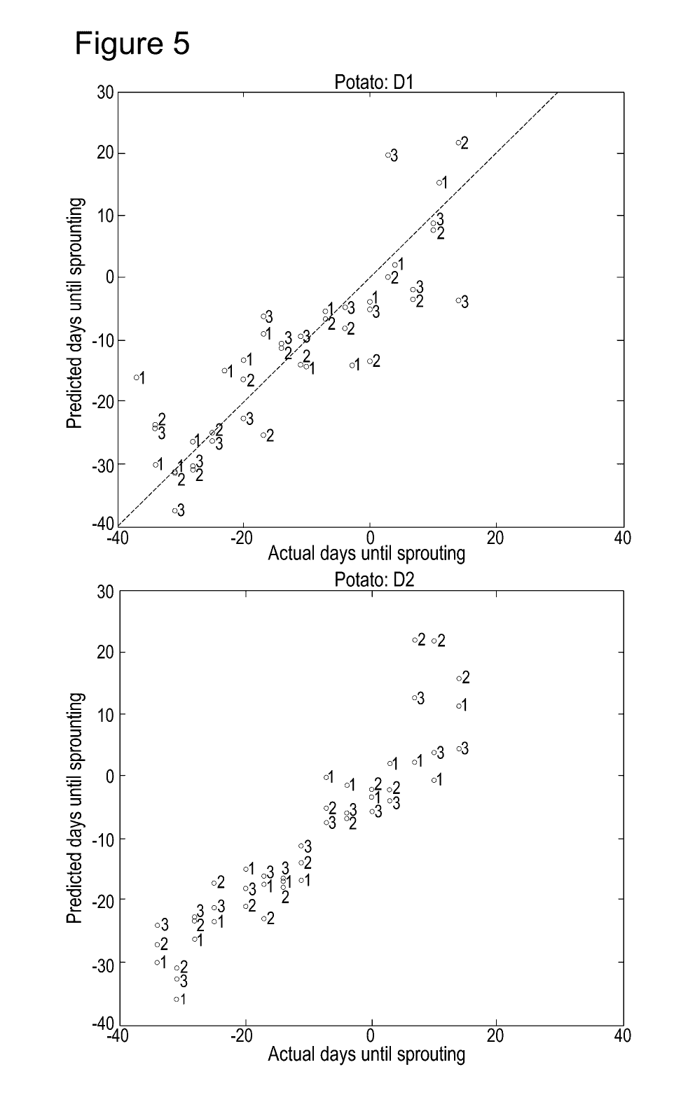

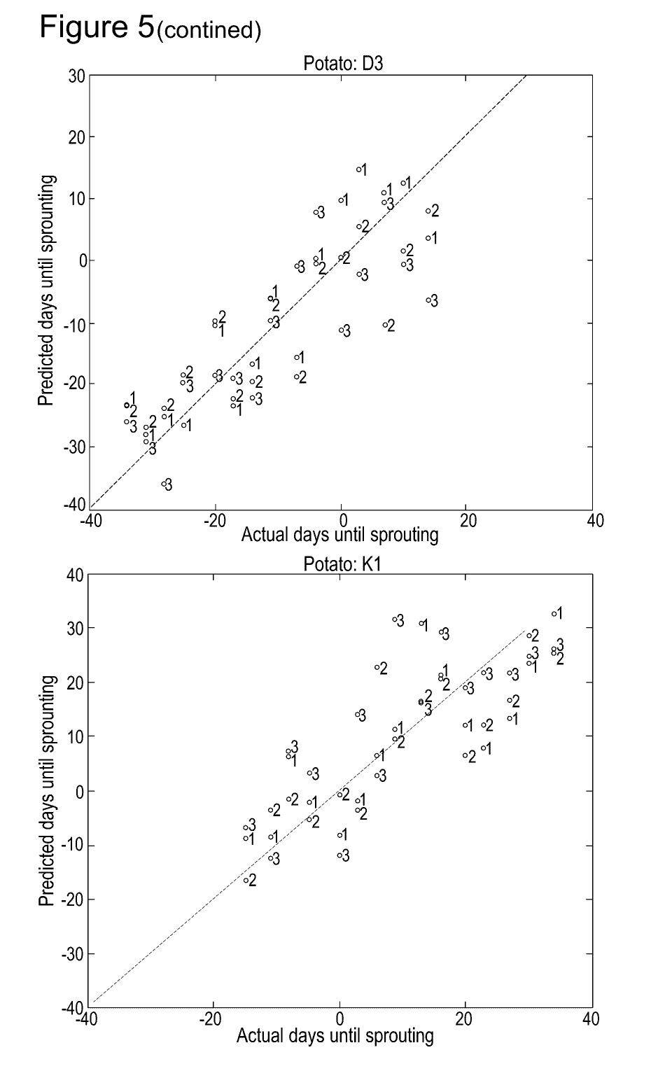

FIG. 5 shows cross-validated predictions obtained by treating each potato separately. It is to be noted that potato “Kind Edward 2” failed to sprout before becoming spoiled due to storage.

As can be seen, the error in prediction for all potatoes was approximately +/−seven days, which represents a commercial useful outcome in practice.

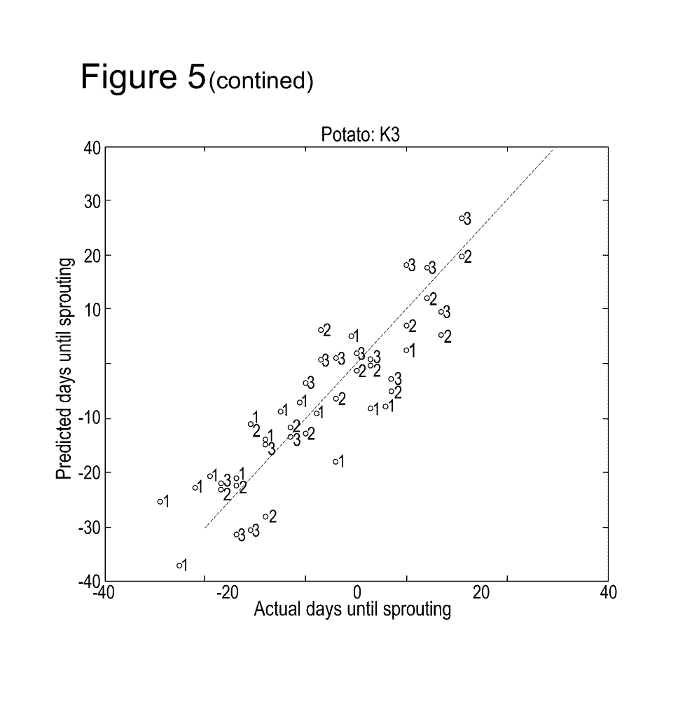

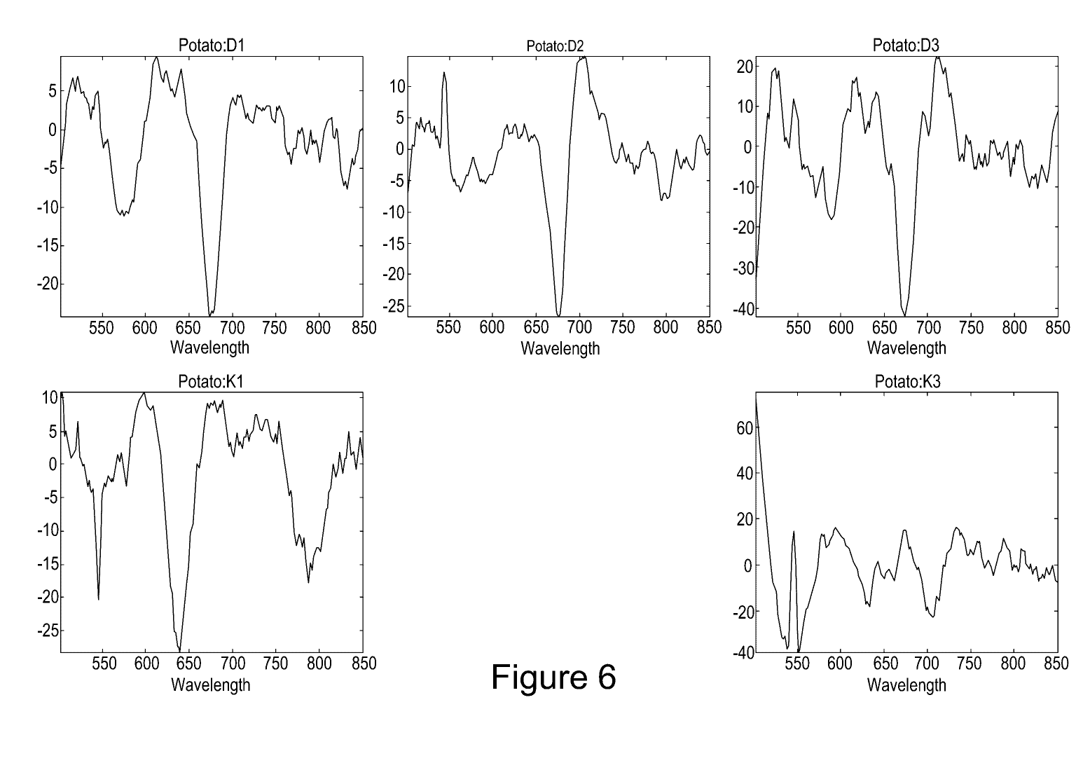

The regression coefficients were examined for each of the Desiree and King Edward potatoes of FIG. 5, and are shown in FIG. 6.

The obtained regression coefficients were very similar, in particular across the Desiree tubers. Furthermore, they were also highly similar to the equivalent regression vector obtained from Maris Piper tubers discussed previously. Specifically, there was a large feature at approximately 675 nm that was negatively associated with increasing age. A predominant feature occurred at 640 nm for the King Edward tubers, wherein the feature was shifted slightly with respect to the one observed for Maris Piper tubers. The skilled person will appreciate that the precise location of the feature is cultivar dependent.

It is important to note that the data from each tuber were treated entirely separately here. The fact that the coefficients were so similar was strong evidence for a common effect across all potatoes.

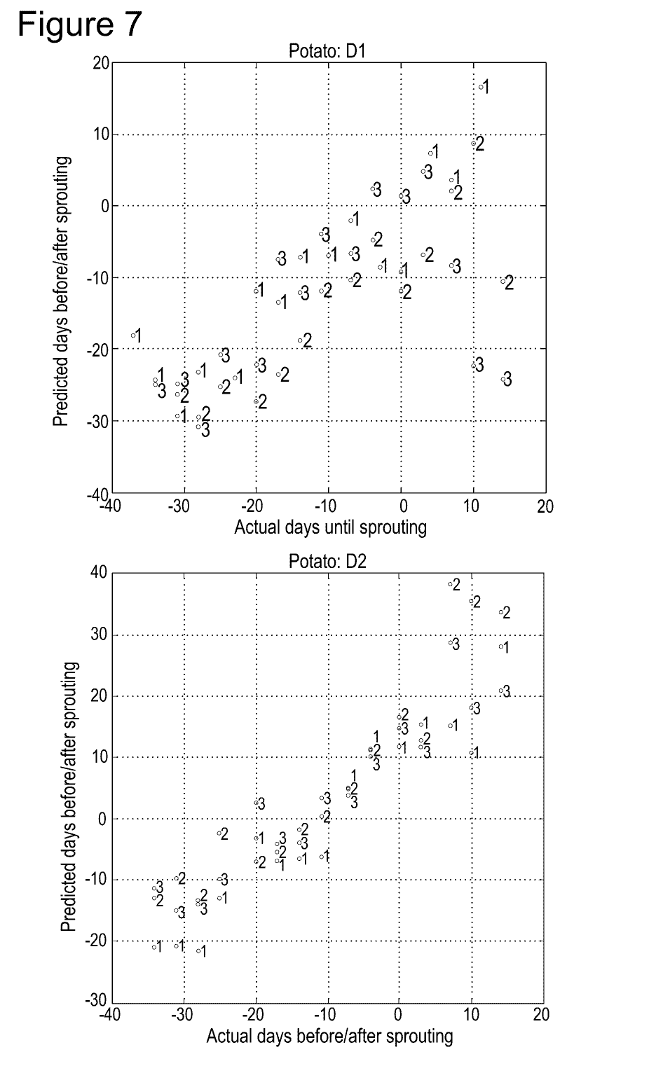

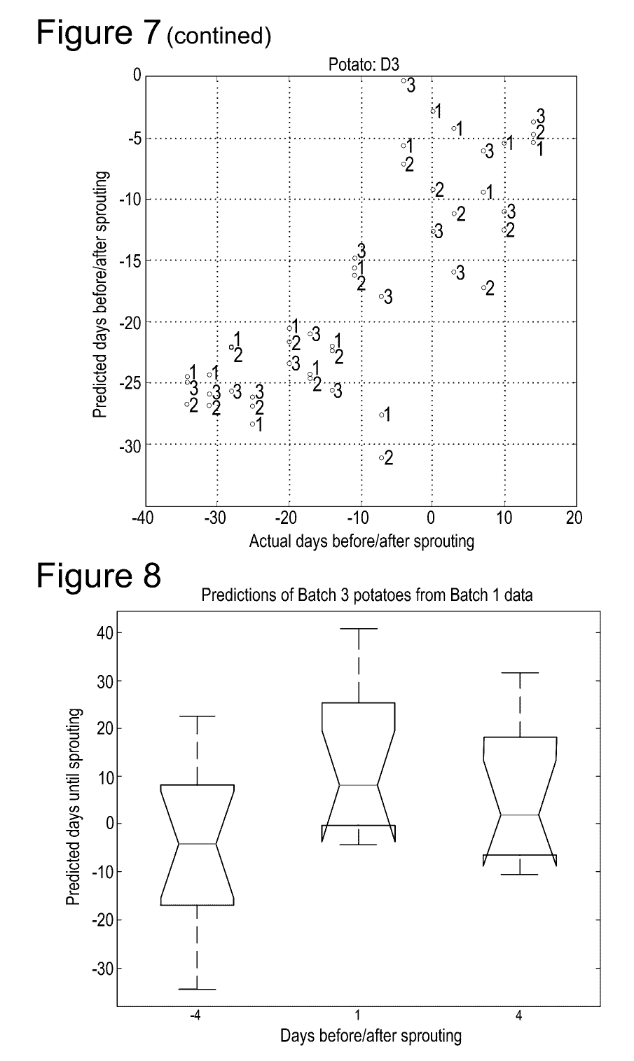

In a further modelling work, leave-tuber-out cross-validation was employed (as for Maris Piper potatoes stored in the dark between analysis), as this more closely mimics the required real-world situation, in which different tubers are used in the model from those that will be subsequently tested. The results are shown in FIG. 7.

The most effective calibrations were obtained from the Desiree tubers, which were able to predict the “left-out” tuber's time-until-sprouting with a precision of approximately +/−seven days.

Two further batches of potatoes were obtained from the same commercial store, at intervals of two months. Unfortunately, the Batch 2 tubers sprouted within a day of arrival at the laboratory, so no proper measurements could be taken in advance of sprouting. Batch 3 tubers also sprouted within a few days of arrival at the laboratory, but one set of measurements were able to be taken in that time.

Where measurements were performed after sprouting, the data was less reliable due to the physical changes in the apical bud area and the consequent reflectance changes. However, this data was used as independent “test” data with which to challenge the models described for the first batch (“Batch 1” above).

FIG. 8 shows a boxplot summary of the predictions for Batch 3 Desiree tubers using a model developed from Batch 1 data.

It can be seen that there was a general trend implying that Batch 1 data could make credible predictions about Batch 3, as seen for the 95% confidence interval around the median prediction for time point −4 days. At −4 days before sprouting, the data predicts 4 days to sprout.

As described above, Batch 2 and Batch 3 tubers sprouted soon after arrival at the laboratory. Hence, the Batch 2 and Batch 3 data in FIG. 8 can be disregarded, since no proper measurements could be taken in advance of sprouting. Hence, it was clear that more tubers exhibiting a longer dormancy period would be needed to carry out proper model validation.

It will be appreciated that not all potatoes always behave in a predictable manner. This is because sampling handling and storage is difficult: any kind of shock (light, temperature change, humidity change, etc.) seems to be able to cause the potatoes to break dormancy suddenly and be driven to sprouting quickly. It may therefore be preferable to analyse potatoes in situ in the potato storage facility.

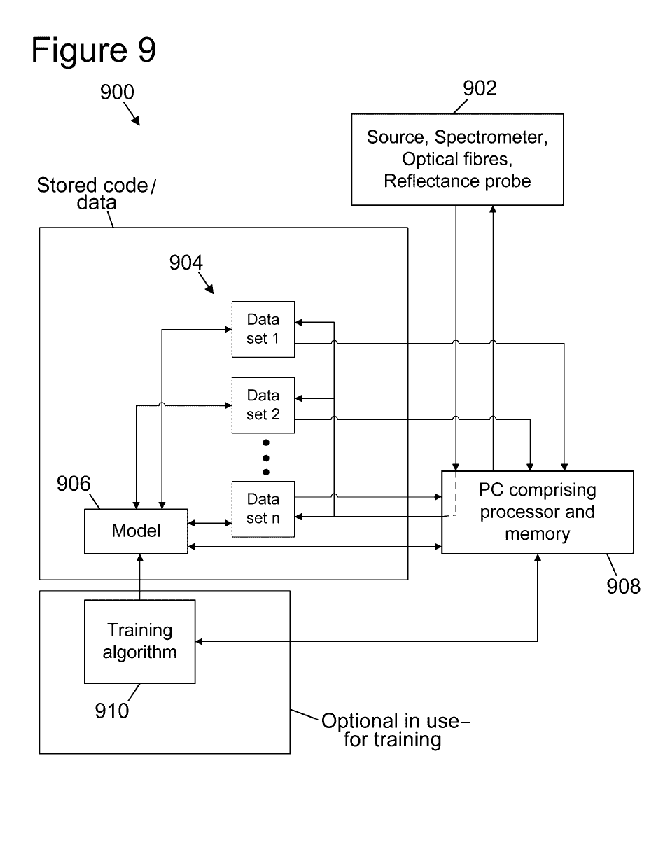

FIG. 9 shows an example experimental setup 900 used in embodiments of this invention. A system 902, which may comprise a light source, a spectrometer, optical fibres and a reflectance probe, is used to measure one or more reflectance spectra. One or more data sets 904 are obtained using the system 902 and may then be further processed and stored in computer 908 comprising a processor and memory.

It will be understood that controlling of system 902 and processing and/or storing data sets 904 may be performed using the same single computer 908, or different computers.

A model 906, as outlined above, which may be stored in memory of computer 908 or a different memory, may then be applied to the one or more data sets 904. The skilled person will appreciate that the model may be applied to the data sets 904 first before the obtained data is input in computer 908 for further processing. Alternatively or additionally, the data sets 904 may first be input and stored in computer 908, and model 906 may be applied to the data sets 904 stored in computer 908 for further processing. In the latter case, it will be appreciated that model 906 may be in direct communication with computer 908, or alternatively, model 906 is stored in memory of computer 908.

It will be understood that one model 906 may be applied per cultivar, i.e. model 906 may be different for, e.g. Kind Edwards tubers, Maris Piper tubers or Desiree tubers.

As a result of the processing in the processor of computer 908, a training algorithm 910 may further be developed. This is an optional feature in use merely for training purposes, and the skilled person will appreciate that this functionality is not essential for the method and system for making a prediction of when a tuber will sprout. Hence, training algorithm 910 may be stored separately from the system comprising computer 908. A separate storing of the training algorithm 910 allows for a remote updating of model 906 in a network.

It will be understood that alternatively, training algorithm 910 may be stored in memory of computer 908 where it is part of the analysis system.

Training algorithm 910 may then be used to train model 906 in order to improve the system and method for making a prediction of when a tuber will sprout.

It will be appreciated that training algorithm 910 may further comprise information, such as, but not limited to, measurement parameters, for example a preferred wavelength of light used which may depend on the cultivar investigated, duration of one or more measurements, or number or intervals of measurements to be taken. The training data may be processed in computer 908 and applied to future measurements.

Data sets 904 and/or training algorithm 910 and/or model 906 may be stored in memory for measurements being performed at a later stage. It will be understood that the memory of computer 908 may be used, or one or more memories outside computer 908 may be exploited to store data sets 904 and/or training algorithm 910 and/or model 906.

### Harvest 2014 Monitoring

Further experiments were conducted on additional tubers obtained from the 2014 harvest. In order to examine the longer NIR wavelengths, two StellarNet spectrometers (with separate Vis and NIR channels) were exploited to increase the wavelength range to 500-2300 nm.

Four different samples of tubers were collected on Jun. 10, 2014 from various locations: King Edward and Mozart tubers were obtained from a first location, and Maris Piper tubers from second and third locations.

The tubers were prepared and analysed using the methods as outlined above. Spectra were collected from four locations on each tuber, a ‘background’ location that was an area of skin free from any apical buds and three separate ‘eye’ locations of three apical buds. For the present harvest, six tubers were studied from each sample to provide a larger data set. Intending to mimic commercial stores, these tubers were kept in a cold, ventilated unit to ensure a stable temperature (˜5.7° C.) and relative humidity (˜80%).

The selected potato tubers were monitored over an 18 week period. The spectral data was firstly analysed using Partial Least Squares regression (PLS). This method was used to demonstrate that Vis/NIR spectra collected from a sample of potato tubers may be used to predict the “sprouting age” of another.

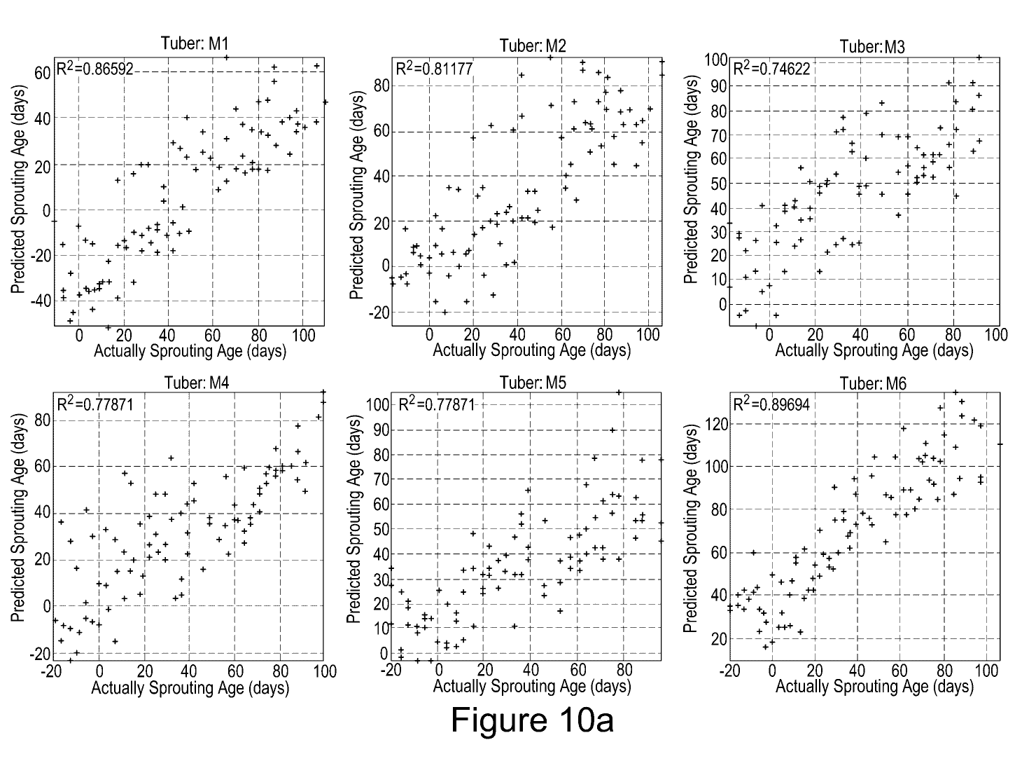

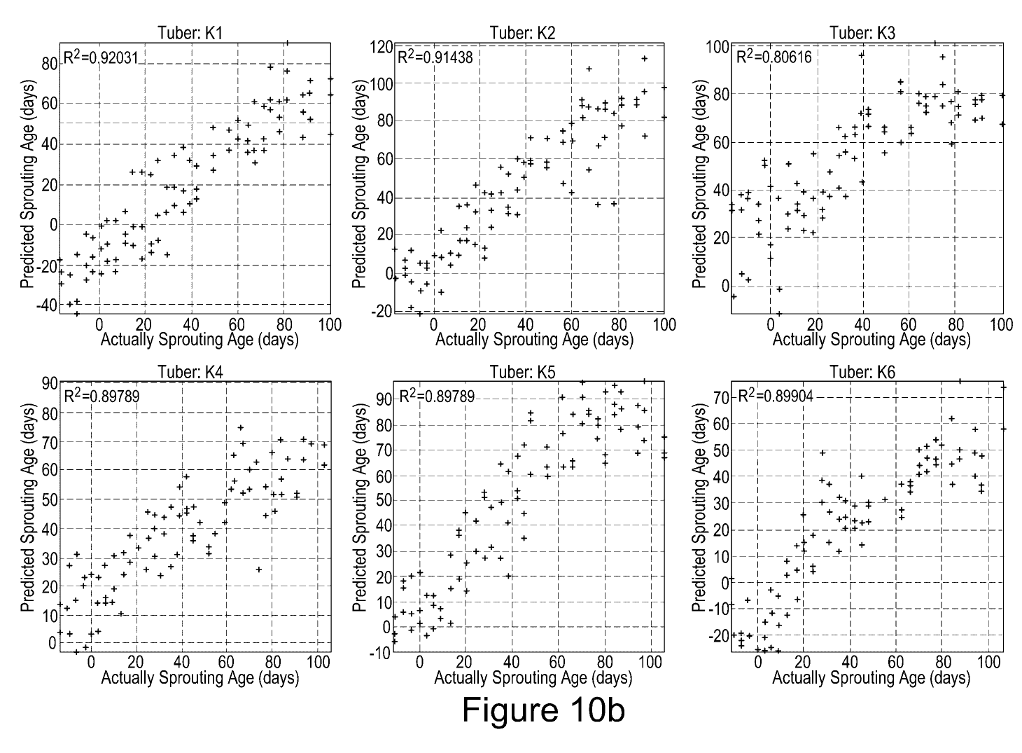

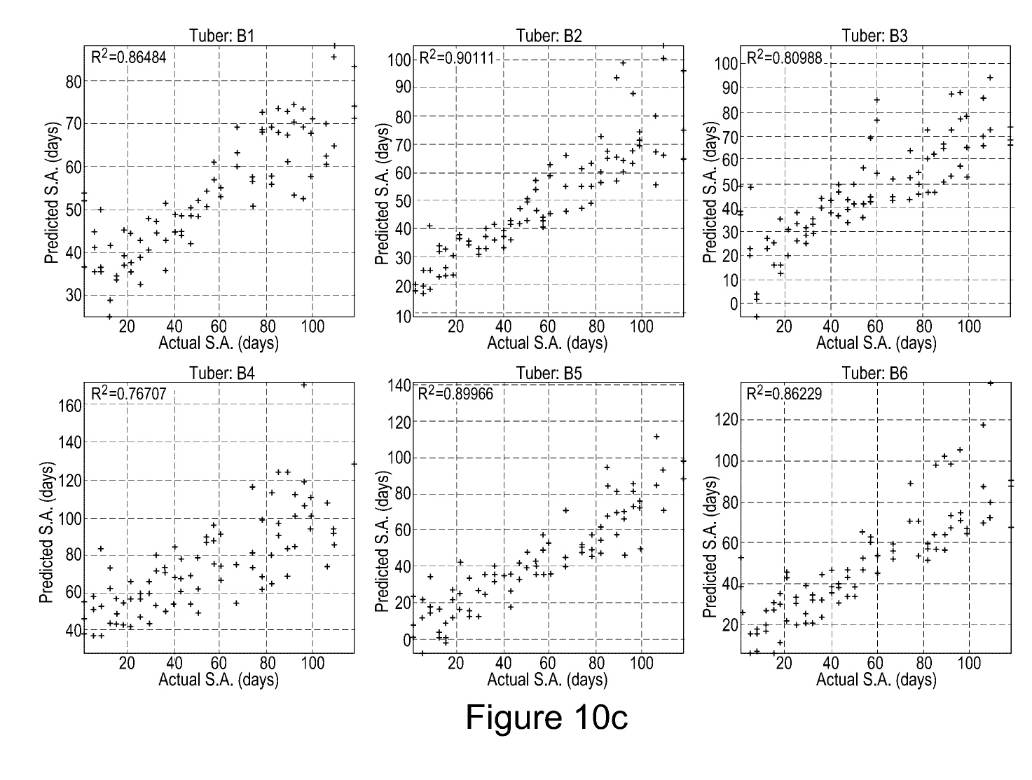

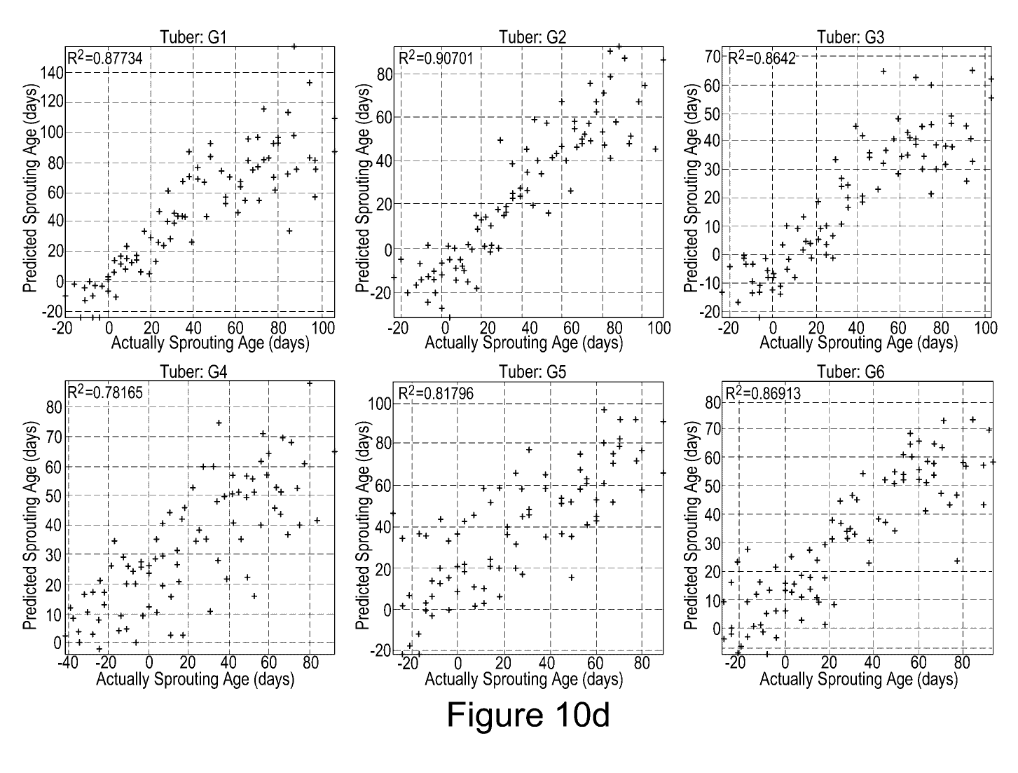

FIG. 10a shows actual sprouting ages (calculated by defining the day on which sprout growth was first visible as day zero) against the predicted sprouting ages calculated by the optimal PLS model for six Mozart tubers. Corresponding graphs are shown in FIG. 10b for six King Edwards tubers, in FIG. 10c for six Maris Piper tubers from a first location, and in FIG. 10d for six Maris Piper tubers from a second location.

It can be seen that very similar features are observed in the figures from analysing King Edwards and Maris Piper tubers in the same way as Mozart tubers. Correlation coefficients between the actual and predicted sprouting ages were found to be as high as 0.92.

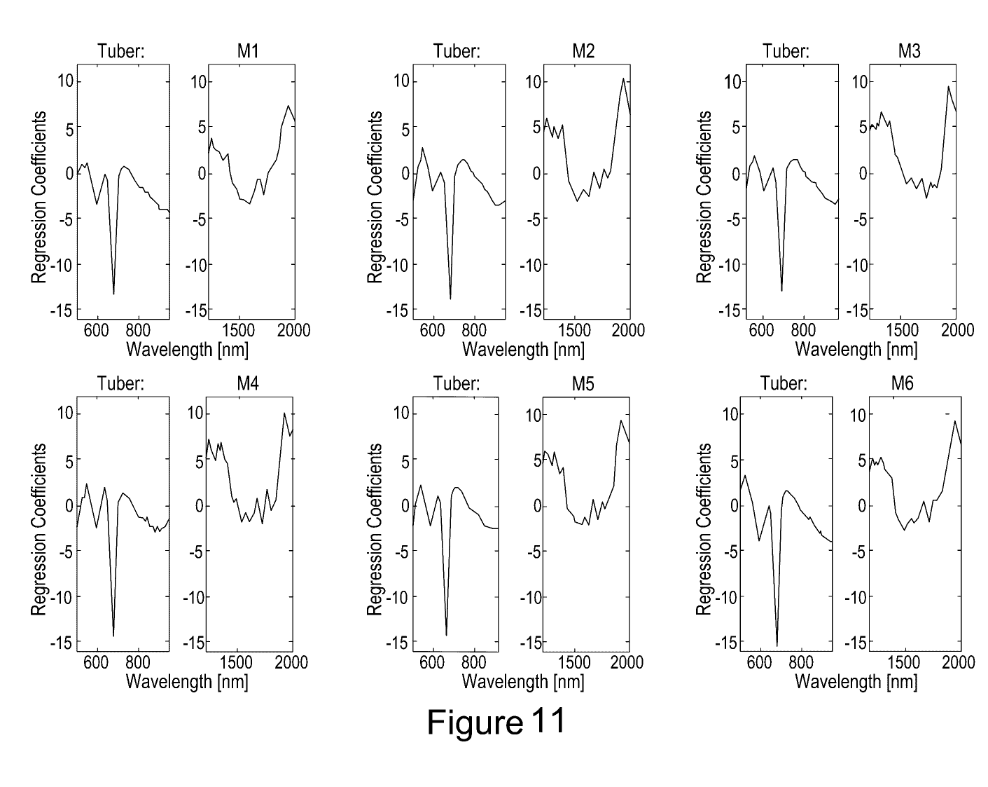

FIG. 11 shows regression coefficients related to the PLS modelling created for the six Mozart tubers analysed as shown in FIG. 10a. In each case, the pair of plots represents the regression coefficients from the two spectrometers (i.e. the separate Vis and NIR channels).

These regression coefficients reiterate the importance of the change in the spectra that occurs between 600-750 nm, evident as the greatest feature in all plots of FIG. 11. The similarity between the six plots of the six separate Mozart tubers stresses that these individual tubers all behaved in a similar way to one another, illustrating that the change in this feature during ageing is common for all tubers.

Due to the extended wavelength range of 500-2300 nm, the experiments specified above, which were conducted over a period of three days, were repeated using a tuber from the 2014 harvest.

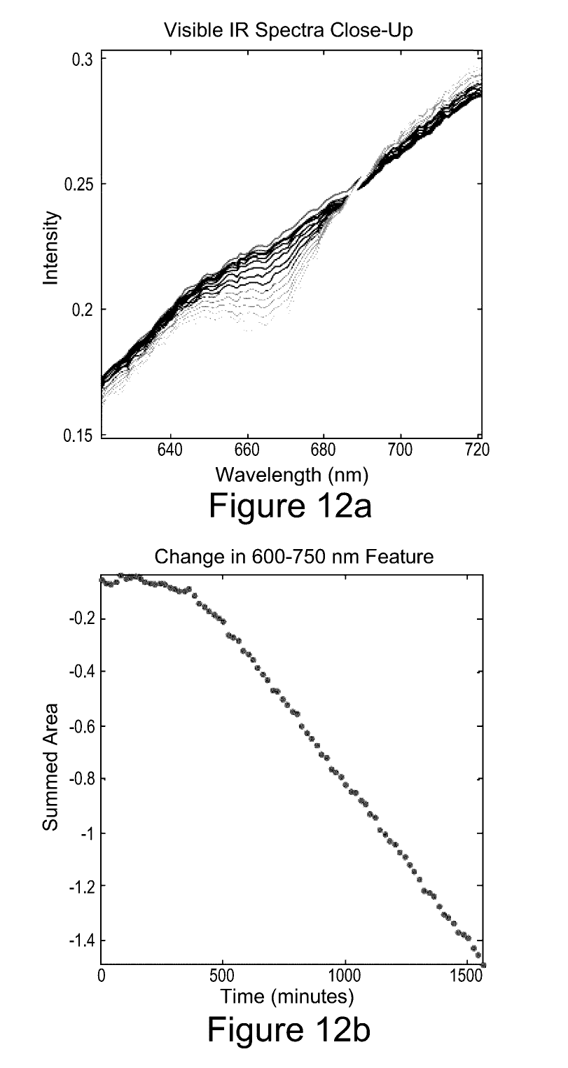

The result is shown in FIG. 12a, which only shows a part of the measured wavelength range (500-2300 nm), in this example from about 620 nm to 720 nm. The curves show the initial spectra (red) taken from an unsprouted tuber at time zero through to the final spectra (cyan) taken from the same tuber, which sprouted a few days later.

As can be seen in FIG. 12a, the largest change in intensity was observed in the 600-700 nm region.

In order to evaluate the change from the initial to the final spectra, a method was applied to calculate the size of the increasingly negative feature between 660 nm and 680 nm. Polynomial curves (from 600 nm to 750 nm) were hereby fitted to each spectrum and then subtracted from the original time zero spectrum.

The result is shown in FIG. 12b, in which the area of these spectra section is calculated and plotted against time. A sudden change occurring in the tuber after approximately 400 minutes was observed.

In order to assess whether the sudden change at around 400 minutes as shown in FIG. 12b may be linked to dormancy breaking, a similar method of data analysis was also performed on the long-term monitoring data of the King Edward, Mozart and Maris Piper tubers.

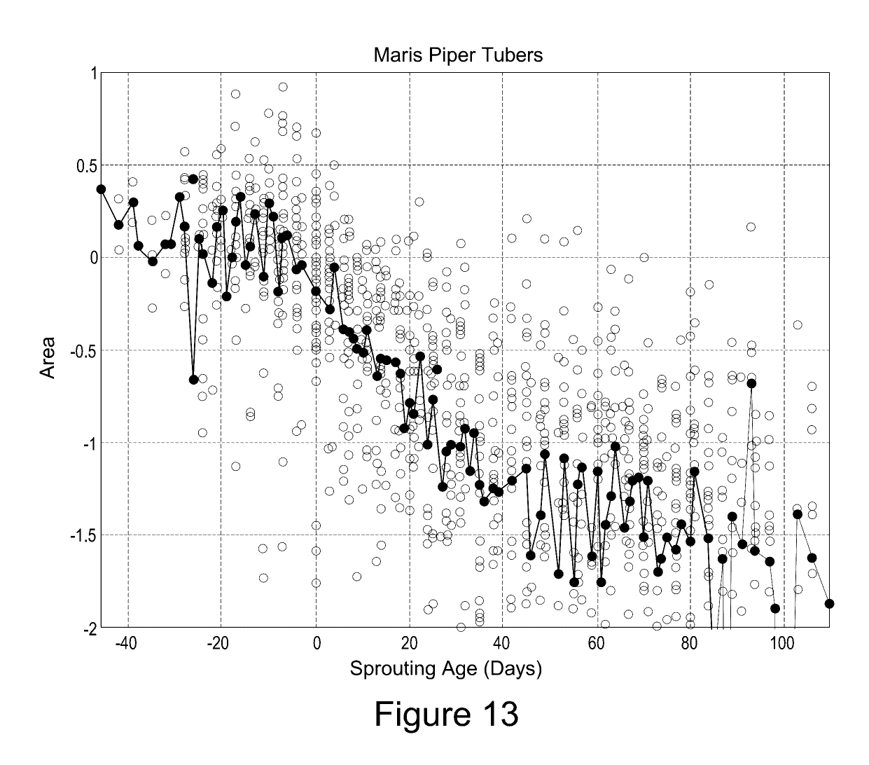

FIG. 13 shows the calculated area difference between each spectra and a related fitted polynomial curve from 600 nm to 750 nm for Maris Piper tubers. In contrast to the single-beam spectra outlined above, the polynomial fitting has here been applied to the eye:background ratio spectra.

The ˜690 nm feature only begins to appear at the point at which the tubers had been recorded to start showing (barely) visible signs of sprouting. From this point onwards, the summed area steadily decreases over time.

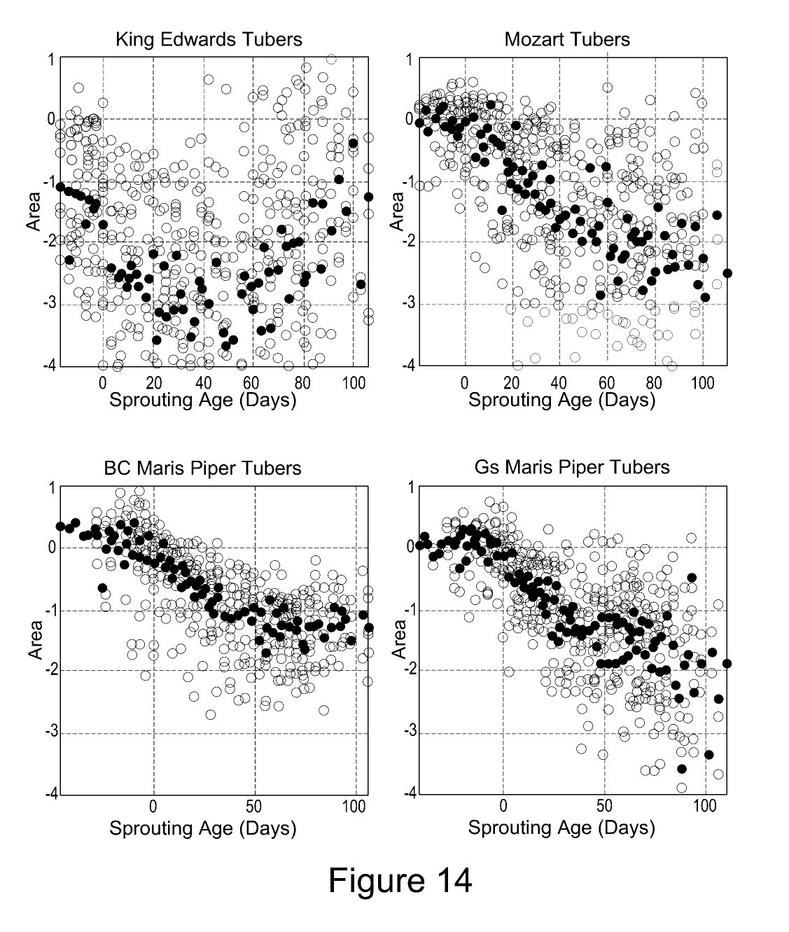

The same analysis as the one shown in FIG. 13 has been applied to various tubers. FIG. 14 shows the calculated area difference versus sprouting age for King Edwards tubers, Mozart tubers, and Maris Piper tubers from two different sources. For this plot, the ratio (eye/background spectra), for each set of tuber samples was used to calculate the area difference between a each spectra and fitted to a polynomial curve from 600 nm to 750 nm.

The analysis displayed in FIG. 14 shows that the results were similar for the Mozart tubers and each of the two Maris Piper tubers. This method analysis may therefore be used as a simple alternative to the PLS regression approaches to predict when tubers are about to break dormancy.

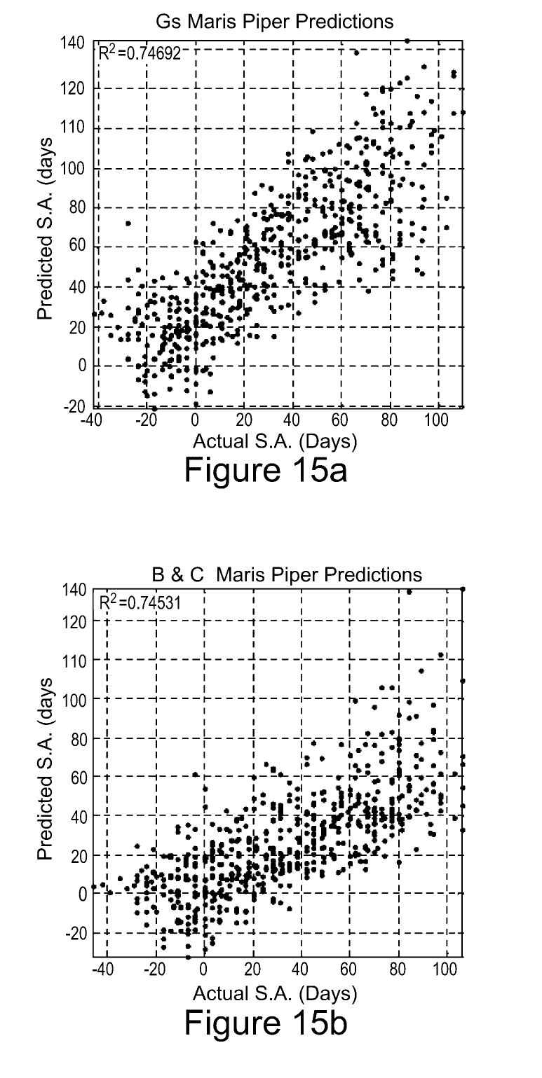

FIGS. 15a and 15b shows predicted sprouting age versus actual sprouting age for different Maris Piper tubers.

For the plot shown in FIG. 15a, PLS predictions were calculated using B & C Farming Maris Piper tubers to create the training set that was used to predict the G's Fresh Maris Piper tubers sprouting ages. For the plot shown in FIG. 15b, G's Fresh tubers were used to obtain a training set to predict sprouting ages of the B & C Farming tubers.

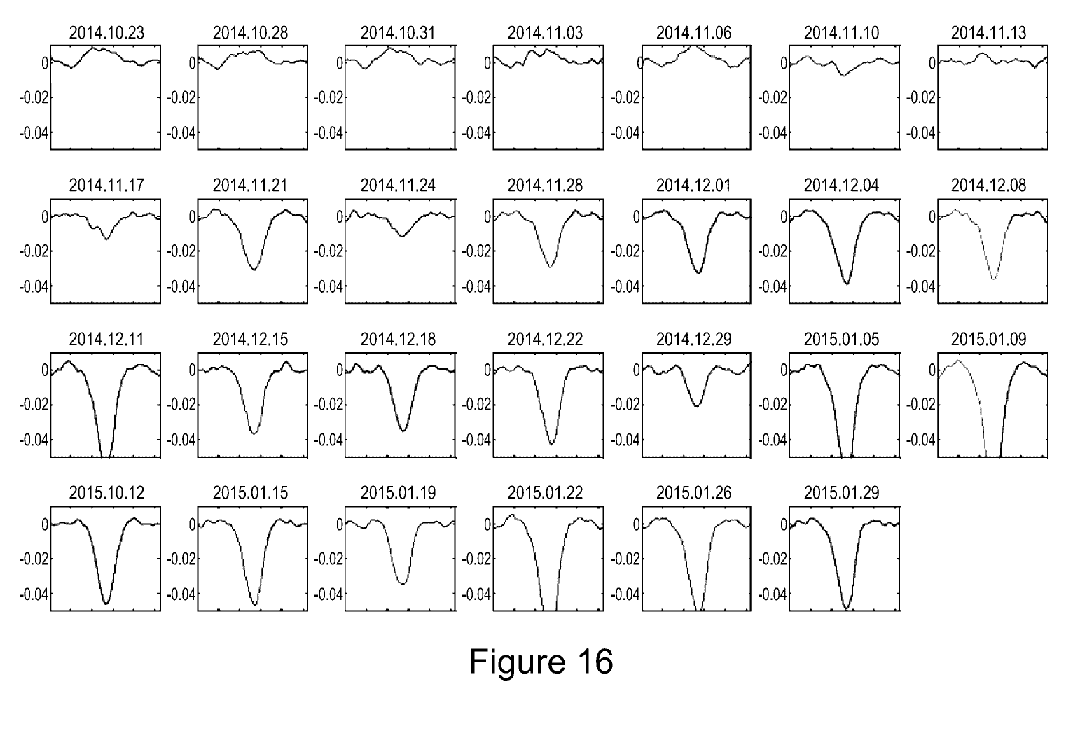

FIG. 16 shows a series of Vis/NIR spectra collected from a single eye and tuber, in this example a Mozart tuber. For clarity, the wavelength labels on the x-axis have been omitted. It can be seen that the ˜690 nm feature changes as a function of date, and generally becomes more pronounced over time.

The above examples show that for separate harvest years and different tuber cultivars, dormancy breaking can be predicted using Vis/NIR spectroscopy. The observed change in the spectral data which occurs between 600 nm and 750 nm (with the central position of the spectral feature concerned varying slightly for different cultivars) allows for predicting sprouting in varies types of tubers.

The PLS modelling applied to the 2014 harvest data set has been shown to be effective for all types of cultivars monitored. As shown, in this example, for Maris Piper tubers from different locations (which may further encompass different farming practices and/or a different local climate), the method allows for predicting one another's sprouting ages, as shown in FIG. 16, reinforcing that the dominant feature between 600 nm and 750 nm is independent of growing conditions.

A further method of analysis may be implemented to investigate more specifically the spectral data between 600 nm and 750 nm, where the change in a baseline-corrected summed area for this section of the spectrum is plotted against time.

The results in this example are very similar for the Mozart and Maris Piper tubers. Once the summed area started to fall below a value of around −0.25, the first signs of tuber sprouting were observed.

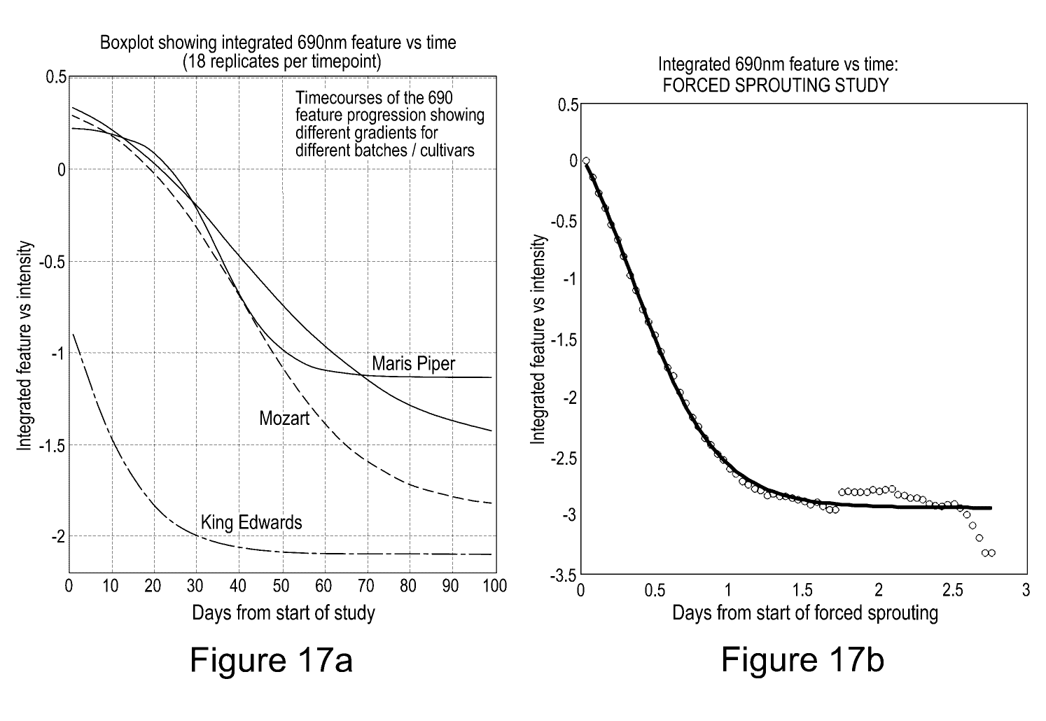

FIG. 17a shows integrated feature intensity of the 690 nm feature versus time (days from the start of the study) for different batches/cultivars of potatoes, in this example from the 2014 harvest. The intensity is, in this example, integrated over a wavelength range, for example from 600 nm to 750 nm. The optical measurements are taken on a tuber eye, and in this example additionally at a non-eye region of the tuber which allows for background correction of the tuber eye measurements.

FIG. 17b shows integrated feature intensity of the 690 nm feature versus time for tubers which were forced to sprout.

As can be seen, the rate at which the change in integrated feature intensity occurs over time, as well as the starting point of the curves, may vary significantly between types of tubers. The gradient is largest, in this example, for King Edwards tubers, and a much larger gradient may be observed for tubers which are forced to sprout compared to naturally aging tubers (note the different x-axis scale of FIGS. 17a and 17b, respectively).

The King Edwards tubers exhibit a lower integrated feature intensity at the start of the measurements (day zero). This shows that the King Edwards tubers already started sprouting prior to day zero of the measurements.

As outlined above, monitoring the feature intensity (or integrated feature intensity where integration is employed) allows for determining a point in time at which intervention of sprouting may be desired. This point in time may be determined by the change in gradient of the (integrated) feature intensity over time, particularly if the gradient is above a threshold. Additionally or alternatively, the point in time at which intervention may be desired may be determined by the (integrated) feature intensity dropping by a threshold value and/or being below a (integrated) feature intensity level.

Monitoring the progression of a batch in this manner may be particularly useful for identifying a point along a time course at which the sprouting may be intervened, e.g. by spraying a suppressant known to those skilled in the art onto the tubers, and/or using temperature control methods, and/or atmospheric control methods, and/or in-field treatments as outlined above.

No doubt many other effective alternatives will occur to the skilled person. It will be understood that the invention is not limited to the described embodiments and encompasses modifications apparent to those skilled in the art and lying within the spirit and scope of the claims appended hereto.

1. A method of making a prediction of when a tuber will sprout, the method comprising:
making a tuber eye measurement of an optical reflectance of said tuber in an eye region of the tuber;
making a reference measurement of optical reflectance of the said or another tuber;
processing said tuber eye measurement in combination with said reference measurement; and
making a prediction of when said tuber will sprout responsive to a result of said processing.

2. A method as claimed in claim 1 wherein said reference measurement comprises a measurement of a non-eye region of the said or another tuber and wherein said processing includes determining a ratio of said tuber eye measurement and said reference measurement.

3. A method as claimed in claim 1 wherein said tuber eye measurement comprises a measurement at a wavelength in the range 600 nm to 750 nm, in particular in the range 650 nm to 700 nm, more particularly at a wavelength of approximately 690 nm.

4. A method as claimed in claim 2 using a wavelength-sensitive optical sensing system to measure features of an optical reflectance spectrum of said eye region of said tuber to make said measurements of said eye region of the same said tuber at different respective wavelengths.

5. A method as claimed in claim 1 comprising making a further optical reflectance measurement on the said or another tuber and compensating for a reference background signal level in said optical reflectance measurements when processing said tuber eye measurement.

6. A method as claimed in claim 1 wherein said processing comprises providing said tuber eye measurement in combination with said reference measurement to a mathematical model providing a prediction of when said tuber will sprout responsive to said tuber eye measurement and said reference measurement, wherein said mathematical model has been trained using a training data set defining measured time to sprouting for a set of said tuber eye measurements and reference measurements.

7. A method as claimed in claim 6 further comprising providing a plurality of said mathematical models, for a plurality of different tuber cultivars, and selecting a said model for making said prediction responsive to a cultivar of said tuber.

8. A method as claimed in claim 6 wherein said model comprises a partial least squares model.

9. A method of identifying a potato or batch of potatoes using the method of claim 1 to make a prediction of when the potato or one or more potatoes of the batch will sprout, and making a decision on sale or use of said potato or said batch responsive to said prediction.

10. A method of processing potatoes using the method of claim 1, the method comprising:
making a tuber eye measurement of an optical reflectance of a potato tuber in an eye region of the tuber;
making a reference measurement of optical reflectance of the said or another tuber;
making a prediction of when said tuber will sprout responsive to said tuber eye measurement in combination with said reference measurement; and
processing said potatoes responsive to said prediction, wherein said processing includes at least separating potatoes predicted to sprout sooner from potatoes predicted to sprout relatively later.

11. A method as claimed in claim 10 further comprising one or both of:
i) storing potatoes selected by said processing in a selected storage area; and
ii) dispatching potatoes selected by said processing.

12. A method as claimed in claim 1, for making a prediction of when a tuber will sprout, the method comprising:
using an optical reflectance measurement system for making a tuber eye measurement of an optical reflectance of an eye region of the tuber, and for making a reference measurement of optical reflectance of the said or another tuber; and
using a data processor, coupled to said optical reflectance measurement system, to process said tuber eye measurement in combination with said reference measurement and to make a prediction of when said tuber will sprout responsive to a result of said processing.

13. A method as claimed in claim 1, wherein making said prediction of when said tuber will sprout comprises identifying a change in gradient of said optical reflectance over time, and/or identifying a reduction in a value of said optical reflectance to below a threshold level or by a threshold amount, and making a prediction of a point of intervention to inhibit said sprouting.

14. A method for predicting sprouting of one or more root vegetables having at least one bud, using the method of claim 1, the method comprising:
measuring a reflectance of at least one of said one or more root vegetables to obtain a reflectance spectrum; and
applying a multivariate model to said reflectance spectrum to predict said sprouting of said one or more root vegetables.

15. A method as claimed in claim 14, wherein said measuring comprises performing a plurality of reflectance measurements at two or more locations on each of said at least one of said one or more root vegetables.

16. A method as claimed in claim 15, wherein said two or more locations are at least one apical bud and at least one location away from an apical bud on said at least one of said one or more root vegetables.

17. A method as claimed in claim 16, wherein said measurement of said reflectance at said at least one location away from an apical bud is used to obtain a reference background reflectance spectrum.

18. A method as claimed in claim 14, wherein said multivariate model is a linear mathematical model.

19. A method as claimed in claim 14, wherein said multivariate model is a Partial Least Squares model.

20. A method as claimed in claim 19, wherein said applying said multivariate model comprises predicting said sprouting using a calculated regression coefficient for a previous said measured reflectance.

21. A method as claimed in claim 14, wherein said measuring comprises measuring said reflectance in a non-contact, non-destructive mode.

22. A method as claimed in claim 14, wherein said reflectance spectrum is obtained for optical wavelengths between 500 nm and 1200 nm.

23. A method as claimed in claim 14, wherein said root vegetables are potato tubers.

24. A method as claimed in claim 14 for predicting sprouting of one or more root vegetables having at least one bud, the method comprising:
using a spectrometer to measure a reflectance of at least one of said one or more root vegetables to obtain a reflectance spectrum; and
using a processor configured to apply a multivariate model to said reflectance spectrum to predict said sprouting of said one or more root vegetables.

25. A method for predicting sprouting of one or more root vegetables, as claimed in claim 14, the method comprising:
measuring a reflectance of at least one of said one or more root vegetables at a plurality of points in time to obtain a plurality of reflectance spectra;
fitting a curve to each of said reflectance spectra; and
comparing said fitted curves to predict said sprouting of said one or more root vegetables.

26. A method as claimed in claim 25, wherein said fitted curves are polynomial curves.

27. A method as claimed in claim 25 further comprising subtracting each of said curves from an initial reflectance spectrum of said plurality of reflectance spectra prior to said comparison.

28. A method as claimed in claim 25 further comprising subtracting each of said measured reflectance spectra from an initial reflectance spectrum of said plurality of reflectance spectra to obtain a plurality of normalised reflectance spectra, and wherein said fitting comprises fitting a curve to each of said normalised reflectance spectra.

29. A method as claimed in claim 25, wherein said measuring comprises measuring a reflectance at an apical bud of a said root vegetable.

30. A method as claimed in claim 1 for monitoring tuber sprouting to predict a point of intervention to inhibit the sprouting, the method comprising:
making a time series of optical measurements on said tuber at at least one wavelength to provide time series spatial data;
processing said time series optical data to monitor the evolution over time of a spectral feature in said optical measurements; and
predicting a point of intervention to inhibit the sprouting, from said evolution over time.

31. A method as claimed in claim 30 comprising making said optical measurements at a plurality of wavelengths or over a wavelength range; wherein said spectral feature is an integrated spectral feature comprising a sum or integration over said plurality of wavelengths or wavelength range; and wherein said processing comprises monitoring the evolution of said integrated spectral feature over time.

32. A method as claimed in claim 30 wherein predicting comprises identifying a change in gradient of said evolution over time.

33. A method as claimed in claim 32 wherein said identifying of said change in gradient comprise identifying a reduction in gradient.

34. A method as claimed in claim 32 wherein said identifying of said change in gradient comprises identifying when
$$\frac{d I}{d t} > C$$
where C is a threshold value less than 0, l represents a value of said spectral feature, and t represents time.

35. A method as claimed in claim 32 wherein predicting comprises identifying a reduction in a value of said monitored spectral feature to below a threshold level or by a threshold amount.

36. A method as claimed in claim 30 wherein said tuber is a potato, wherein said at least one wavelength is a wavelength in the range of 600 nm to 750 nm, and wherein said optical measurements comprise measurements of an eye of said potato.

37. A method as claimed in claim 30 further comprising inhibiting said sprouting by applying a sprouting suppressant to said tuber in response to said predicting.

38. Apparatus for determining the condition of a tuber, the system comprising:
a light source to stimulate the tuber to promote chlorophyll production and/or sprouting, wherein said light source is configured to provide substantially continuous optical stimulation to the tuber;
an optical instrument to measure an optical response of the tuber;
a controller to control said optical instrument to make a time series of optical measurements on said tuber at intervals during said substantially continuous optical stimulation to determine a time evolution of an optical response of the tuber, wherein said time series evolution is determined over a period of less than twenty four hours;
a data processor to analyse said time evolution of said optical response to determine a condition of the tuber.

39. Apparatus as claimed in claim 38 wherein said condition of said tuber comprises a prediction of when said tuber will sprout or defines a sprouting propensity of said tuber.

40. Apparatus as claimed in claim 39 wherein said data processor is configured to analyse said optical response to determine a condition of the tuber by determining one or more of i) a time interval until a threshold change in said optical response; ii) a rate of change of said optical response; and iii) a curve fit to said optical response.

41. Apparatus as claimed in claim 39 wherein said optical response comprises an optical reflectance or absorption response in the range 600 nm to 750 nm and/or an integrated optical response over a wavelength band.

42. Apparatus as claimed in claim 39 wherein said optical response comprises an optical reflectance or absorption response within 50 nm of an absorption band of chlorophyll.

43. Apparatus as claimed in claim 38 wherein said time series evolution is determined over a period of less than one hour.

44. Apparatus as claimed in claim 38 wherein said controller is configured to control said optical instrument to make measurements at least every 5 minutes.

45. A method of determining the sprouting propensity of a tuber, the method comprising:
applying substantially continuous stimulation to an eye region of the tuber to drive said eye region of the tuber to develop at a faster than natural rate;
making a time series of optical measurements on said eye region of said tuber at at least one wavelength to provide time series optical data to determine a time evolution of an optical response of said eye region of the tuber during said stimulation, wherein said time series evolution is determined over a period of less than twenty four hours; and
determining a sprouting propensity of the tuber from said time evolution of said optical response.

46. A method as claimed in claim 45 wherein said substantially continuous stimulation comprises light.

47. A method as claimed in claim 46 wherein said development of said eye region of the tuber comprises chlorophyll production in said eye region of the tuber; wherein said sprouting propensity comprises a prediction of when said tuber will sprout, and wherein said determining of said sprouting propensity comprises determining a speed of response of said eye region of said tuber to said stimulation.

48. A method as claimed in claim 45 wherein said determining of said sprouting propensity comprises determining one or more of i) a time interval until a threshold change in said optical response; ii) a rate of change of said optical response; and iii) a curve fit to said optical response.

49. A method as claimed in claim 45 wherein said time series evolution is determined over a period of less than one hour.
